(19) United States
(12) Patent Application Publication
(10) Pub. No.: US 2015/0346202 A1
(43) Pub. Date: Dec. 3, 2015

(54) STABILIZED CHEMILUMINESCENT SYSTEM

(71) Applicant: Ll-COR, INC., Lincoln, NE (US)

(72) Inventors: Kousik Kundu, Lincoln, NE (US); Ying Wang, Lincoln, NE (US); Nisha Padhye, Lincoln, NE (US)

(73) Assignee: Ll-COR, INC., Lincoln, NE (US)

(21) Appl. No.: 14/724,063

(22) Filed: May 28, 2015

Related U.S. Application Data
(63) Continuation of application No. PCT/US2015/032641, filed on May 28, 2015.
(60) Provisional application No. 62/004,151, filed on May 28, 2014.

(51) Int. Cl. G01N 33/573

(52) U.S. Cl. CPC G01N 33/573; G01N 2333/908

(57) ABSTRACT

The present invention relate to formulations, systems, kits and methods for chemiluminescence assays to detect the catalytic activity of a peroxidase. The present invention provides formulations and methods for chemiluminescence reactions that have significantly improved signal duration, which signal lasts for hours.

### CROSS-REFERENCE TO RELATED APPLICATIONS

This application is a continuation of PCT/US2015/032641 filed May 27, 2015, which application claims priority to U.S. Provisional Patent Application No. 62/004,151, filed May 28, 2014, the teachings of which are hereby incorporated by reference in their entirety.

### BACKGROUND OF THE INVENTION

Electrochemiluminescence or electrogenerated chemiluminescence (ECL) is the luminescence produced during chemical reactions. ECL has been widely used for ultrasensitive detection of proteins by Western blot (WB) analysis. One major advantage of ECL is high sensitivity due to very low background. Luminol-based chemiluminescence substrates for HRP-based Western blot analysis have been very effective, mainly due to the development of highly effective enhancers i.e., thiazines. These enhancers significantly improve the sensitivity of low protein concentrations. Therefore, the enhancers play a key role in the chemiluminescence Western blot analysis.

Two major classes of enhancers have been developed in the last 2-3 decades i.e., phenols and N-alkyl thiazines. N-alkyl thiazines have shown good performance in terms of signal duration and sensitivity. Phenols, on the other hand, although they show good sensitivity during the initial time points, suffer from signal duration and long-term stability. Typically, the signal drops so fast that the light intensity becomes negligible within 10-20 min or even less.

Such a quick loss of chemiluminescence intensity leads to irreproducibility of data, difficulty of use, a higher chance of creating artifacts, incompatibility with different chemiluminescence detection systems and incoherent responses. The current phenol based enhancer systems therefore have many limitations.

In view of the foregoing, there is a need to develop a phenol based chemiluminescence system with longer lifetime intensity. A longer lifetime will result in reproducibility of results, ease of use, and less of a chance for creating artifacts. The present invention satisfies these and other needs.

### BRIEF SUMMARY OF THE INVENTION

The present invention relates to chemiluminescent systems, methods and formulations and especially enhanced chemiluminescent systems and formulations useful in assays of peroxidase activity. As such, in one embodiment, the present invention provides a formulation for chemiluminescence, the formulation comprising, consisting essentially of or consisting of, a first part and a second part:
a) the first part comprising:
   i) a diacylhydrazide selected from the group consisting of luminol, isoluminol or a luminol derivative;
   ii) a phenol enhancer;
   iii) optionally one or more co-enhancers;
   iv) one or more stabilizers; and
b) the second part comprising:
   v) an oxidant.

In another embodiment, the present invention provides a method of light emission from a chemiluminescence formulation, the method comprising: providing a formulation for chemiluminescence, the formulation comprising, consisting essentially of, or consisting of: a first part and a second part:
a) the first part comprising:
   i) a diacylhydrazide selected from the group consisting of luminol, isoluminol or a luminol derivative;
   ii) a phenol enhancer;
   iii) optionally one or more co-enhancers;
   iv) one or more stabilizers; and
b) the second part comprising:
   v) an oxidant, in the presence of a peroxidase to provide light emission from a chemiluminescence formulation.

The present invention provides formulations, methods and systems for a chemiluminescent assay of peroxidase activity that is useful in connection with the detection of analytes of all types (e.g., biological molecules, organic molecules, natural or synthetic molecules). The invention is particularly applicable to detection of proteins and nucleic acids using all types of membrane-based assays by techniques such as Western blotting, Dot blotting, Southern blotting, and Northern blotting. Furthermore, the present invention is particularly applicable to the detection of analytes using all types of solution-based, luminometric assays, such as ELISAs (Enzyme Linked Immunoabsorbent Assays), bead assays, and the like.

In still yet another embodiment, the present invention provide a kit for chemiluminescence, the kit comprising, consisting essentially of, or consisting of a first part and a second part:
a) the first part comprising:
   i) a diacylhydrazide selected from the group consisting of luminol, isoluminol or a luminol derivative;
   ii) a phenol enhancer;
   iii) optionally one or more co-enhancers;
   iv) one or more stabilizers; and
b) the second part comprising:
   v) an oxidant, in the presence of a peroxidase to provide light emission from a chemiluminescence formulation and instructions for use.

These and other aspects, objects and advantages will become more apparent when read with the drawings and detailed description which follow.

### BRIEF DESCRIPTION OF THE DRAWINGS

FIGS. 1A-D show images of Western blots treated with the SuperSignal West Dura (“Dura”) chemiluminescent substrate or the test substrate containing the phenol enhancer BIPCA. Signal intensity and sensitivity of the Dura treat blots at the 2 minute acquisition time point (FIG. 1A) and at the 30 minute time point (FIG. 1C) can be compared to that of the test substrate blots at the 2 minute time point (FIG. 1B) and at the 30 minute time point (FIG. 1D).

FIGS. 2A-C show images of Western blots incubated with the Dura substrate, SuperSignal West Pico (“Pico”) substrate or test substrates #27-30 over time. FIG. 2A shows the images at time 2 minutes. FIG. 2B shows the images at 30 minutes and FIG. 2C shows the images at time 1 hour.

FIG. 3A shows the experimental design of the experiment to test substrates #49A-49D on slot blots. FIG. 3B shows the formulations of the test substrates. Slot blots of HRP labeled Goat anti-Mouse (1 ng→125 fg) spotted on Odyssey Nitrocellulose Membrane in triplicate. The blots were blocked in 5% Skim Milk for 1 hr; and incubated for 5 minutes in various Chemiluminescent Substrates. The images were acquired on the Odyssey Fc at 2 min exposures.

FIGS. 4A-B show images of slot blots treated with the test substrates #49A-49D, Pico substrate or Dura substrate at time 0 (FIG. 4A) or at time 1 hour (FIG. 4B).

FIG. 5A shows the experimental design of the experiment to test substrates #B1 and F12 on slot blots. FIG. 5B shows the formulations of the test substrates. Slot blots of HRP labeled Goat anti-Mouse (1 ng→125 fg) spotted on Odyssey Nitrocellulose Membrane in triplicate. The blots were blocked in 5% Skim Milk for 1 hr; and incubated for 5 minutes in various Chemiluminescent Substrates. The images were acquired on the Odyssey Fc at 2 min exposures.

FIGS. 6A-B show images of slot blots treated with the test substrates #B1 and F12, Pico substrate or Dura substrate at time 0 (FIG. 6A) or at time 1 hour (FIG. 6B).

FIG. 7A shows the experimental design of the experiment to test substrates ##6-5-D, 6-5-E, 6-5-H, and 6-5-J on slot blots. FIG. 7B shows the formulations of the test substrates.

FIGS. 8A-B show images of slot blots treated with the test substrates, Clarity™ substrate or Dura substrate at time 0 (FIG. 8A) or at time 1 hour (FIG. 8B).

FIGS. 9A-C show images of Western blots treated with the test substrate containing BIPCA, urea and DMPA. The images were acquired on the C-DiGit blot scanner as three successive 12 minute scans. FIGS. 9A, 9B, and 9C represent the first, second and third scans, respectively. The quantitative data of scans 1, 2, and 3 as well as the control blots are provided in FIGS. 9D, 9E, and 9F, respectively.

FIGS. 10A-B show images of Western blots treated with the test substrate containing BIPCA, urea and DMPA (FIG. 10A) and the control substrate (FIG. 10B). FIGS. 10C-E show the quantitative data at time 0 (FIG. 10C), 30 minutes (FIG. 10D), and 60 minutes (FIG. 10E) for the test substrate “LI-COR” and the control SuperSignal West Femto (“Femto.”) Western Blot of C32 lysate (10 μg/well→1.2 ng/well) probed for Actin mAb (1:1 k) detected with HRP-GAM (1:5 k) Blots were incubated 5 min in Femto and LICOR Substrates and imaged on the Odyssey Fc (2 min acquisition) at Time 0, 30 min, 1 hr, and 2 hrs. Images are not linked, they are individually optimized.

FIGS. 11A-D show images of Western blots treated with the test substrate containing BIPCA, urea and DMPA (FIG. 11C) and the control substrates: Femto (FIG. 11A), Dura (FIG. 11B) and Pico (FIG. 11D). Western Blot of NIH/3T3 lysate (2.5 μg→69 ng/well) probed for Erk mAb (1:1 k) detected with HRP-GAM. Blots were incubated 5 min in various Chemi Substrates and imaged on the Odyssey Fc (2 min acquisition). Images are not linked, they are individually optimized.

FIG. 12 shows a graph of the quantitative data of the blots in FIG. 11A-D.

FIGS. 13A-D show the blots from the rescue blot experiment. Blot 1 was treated with the control substrate Pico (FIG. 13A) and then with the control substrate Femto (FIG. 13B) after the initial signal was quenched. Similarly, blot 2 was first treated with the control substrate Pico (FIG. 13C) and then with the test substrate (FIG. 13D).

FIGS. 14A-F show images of Western blots treated with a control substrate Femto (FIG. 14A), a test substrate containing a phenol enhancer (FIG. 14B), and a test substrate containing a phenol enhancer and palladium (FIG. 14C). The images were acquired at various time points, e.g., time 0 (1401), 15 minutes (1410), 30 minutes (1420), 1 hour (1430), and 2 hours (1440). FIG. 14D shows a test substrate containing a phenol enhancer in the absence of Mn or Pd; whereas FIGS. 14E and 14F show a test substrate containing a phenol enhancer and either Pd or Mn, respectively.

FIG. 15 shows the remaining chemi-signal after blot incubation with three substrates containing different concentrations of boric acid.

FIG. 16 shows a chemi-signal and the remaining after incubation with substrates with and without Na₃VO₄.

FIG. 17A-D shows a comparison of pyridine derivatives (FIG. 17A-C) as stabilizers compared to a commerically available product (FIG. 17D).

### DETAILED DESCRIPTION OF THE INVENTION

### I. Definitions

The terms “a,” “an,” or “the” as used herein not only include aspects with one member, but also include aspects with more than one member.

The term “about” as used herein to modify a numerical value indicates a defined range around that value. If “X” were the value, “about X” would indicate a value from 0.9X to 1.1X, and more preferably, a value from 0.95X to 1.05X. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and provide written description support for a claim limitation of, e.g., “0.98X.”

The term “assay” includes the detection or quantification of an analyte. Typically, the implementation of an assay requires a correlation of the light output to the amount of peroxidase used, so that peroxidase is the substance determined directly. Although the present invention is useful for determining the presence or amount of any of a reactant (luminol, peroxidase or oxidant), the reactant is not necessarily the substance itself to be determined. For example, the oxidant (e.g., H₂O₂) can be produced by a previous reaction, or a series of previous reactions.

The term “Lewis Acid” includes a substance that can accept a pair of nonbonding electrons. A Lewis acid is an electron-pair acceptor.

The term “oxidant” includes substances which are oxidizing agents or a substance which accepts an electron from another species. An oxidant gains electrons during a reaction and is reduced.

The term “phenol enhancer” includes compounds which are aromatic alcohols.

The term “thiol” includes compounds that contains a carbon-bonded sulfhydryl (R—SH) group.

The term “tertiary amine” includes compounds having all three hydrogen of a nitrogen molecule replaced with an aromatic or aliphatic group, i.e., N—(R¹)(R²)(R³), wherein each R group can be the same or different.

### II. Embodiments

The present invention relates in-part to formulations, systems and methods for the chemiluminescence assays to detect the catalytic activity of a peroxidases. In one aspect, the present invention provides formulations and methods for chemiluminescence reactions that have significantly improved signal duration, which signal lasts for a significant time period e.g., 0.5, 1, 2, 3 or more hours. The formulations and methods provide a flattened signal decay curve compared to prior art enhancers. The longer detection window significantly improves the reproducibility of data, and compatibility with different chemiluminescence detection systems (i.e., films, CCD imagers, and the like). The formulations, methods and systems are useful as chemiluminescence substrates for Western blot applications and other biological assays.

In one embodiment, the present invention provides a formulation for chemiluminescence, the formulation comprising a first part and a second part:
a) the first part comprising:
   i) a diacylhydrazide selected from the group consisting of luminol, isoluminol or a luminol derivative;
   ii) a phenol enhancer;
   iii) optionally one or more co-enhancers;
   iv) one or more stabilizers; and
b) the second part comprising:
   v) an oxidant.

In certain aspects, the first part and the second part are in separate containers, such as in a kit, and admixed prior to use. In certain aspects, the diacylhydrazide is luminol or a luminol derivative. The diacylhydrizide is present in the first part at about 0.1 mM to about 20 mM. In certain aspects, the diacylhydrizide is present at about 1 mM to about 10 mM such as about 1, 2, 3, 4, 5, 6, 7, 8, 9, or about 10 mM. In certain aspects, luminol is present at about 5 mM to about 10 mM such as about 5, 6, 7, 8, 9 or 10 mM.

A wide variety of phenol enhancers are suitable for use in the present invention. In one aspect, the phenol enhancer is selected from the group of 4-indophenol, 4-iodophenol, 4-(3-thienyl)phenol, 4-(1-pyrrolyl)phenol, 4-(4-tolyl)phenol, 4-carboxy-4-hydroxybiphenol (BIPCA), 4-bromo-4-hydroxyphenol or a combination thereof. In a preferred aspect, the phenol enhancer is 4-carboxy-4-hydroxybiphenol or 4′-hydroxy-[1,1′-biphenyl]-4-carboxylic acid (BIPCA), which has the following structure:

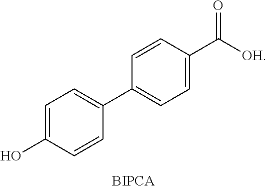 [SMILES: O=C(O)c1ccc(-c2ccc(O)cc2)cc1]

In certain aspects, the present invention provides suitable enhancers including, 4′-hydroxy-[1,1′-biphenyl]-4-carboxylic acid, (E)-3-(4-hydroxyphenyl)acrylic acid, 4-(1H-imidazol-1-yl)phenol, 2-(4′-hydroxy-[1,1′-biphenyl]-4-ylcarboxamido)ethanesulfonic acid, 3-(4′-hydroxy-[1,1′-biphenyl]-4-ylcarboxamido)propane-1-sulfonic acid, 4′-hydroxy-[1,1′-biphenyl]-4-sulfonic acid, 5-hydroxy-1-naphthoic acid, 2-hydroxybenzoic acid, benzo[d]oxazol-6-ol, benzo[d]thiazol-6-ol, and (E)-4-hydroxy-3-(3-(4-hydroxyphenyl)acryloyl)-2H-chromen-2-one. Other enhancers include, but are not limited to, 4-(1,2,4-triazol-1-yl) phenol, 4-phenylphenol, 4-(4,5-diphenyl-1H-imiazole)phenol or 4-(2-methyl-4-thiazolyl)phenol.

In certain other aspects, the phenol enhancer is present in the first part at about 0.01 mM to about 10 mM. In other aspects, the phenol enhancer is present at about 0.1 to about 1.0 mM or to about 5 mM or 0.1, 0.2, 0.3, 0.4, 0.5, 0.6, 0.7, 0.8, 0.9, or about 1.0 mM. In certain aspects, BIPCA is present at about 0.1 mM to about 1.0 mM such as about 0.1, 0.2, 0.3, 0.4, 0.5, 0.6, 0.7, 0.8, 0.9, or about 1.0 mM, or even 1, 2, 3, 4, or 5 mM.

A wide variety of oxidants are suitable for use in the present formulations and methods. Suitable oxidants include a perborate or a peroxide. For example, the perborate can be sodium perborate (NaBO₃) or potassium perborate (KBO₃). The peroxide can be hydrogen peroxide (H₂O₂). In fact, any suitable substrate for a peroxidase will work, for example, urea H₂O₂. Those of skill in the art will know of other oxidants such as other perborates and peroxides suitable for use in the present invention. In certain aspects, the oxidant is present in the second part at about 0.1 mM to about 20 mM. In certain aspects, the oxidant is present at about 1 mM to about 10 mM such as about 1, 2, 3, 4, 5, 6, 7, 8, 9, or about 10 mM. In certain other aspects, sodium perborate is present at about 1 mM to about 10 mM such as about 1, 2, 3, 4, 5, 6, 7, 8, 9 or 10 mM.

In certain aspects, the first part of the formulation comprises a buffer. The buffer maintains the pH of the first part between a pH of about 6 to about 12, or about 6-8, 6-9, 6-10, 6-11, 7-8, 7-9, 7-10, 7-11, 7-12, 8-9, 8-10, 8-11, 8-12, 9-10, 9-11, 9-12, 10-11, 10-12, 6, 7, 8, 9, 10, 11, or a pH of about 12. In one aspect the buffer of the first part is a Tris buffer. The Tris buffer can have a concentration of about 50 mM to about 1000 mM, such as about 100, 200, 300, 400, 500, 600, 700, 800, 900 or 1000 mM (1.0 M).

In certain aspects, the second part comprises a buffer. In a preferred aspect, the buffer of the second part has a pH of about 3 to about 7, or about 3 to about 10 such as about 3, 4, 5, 6, 7, 8, 9 or 10, or 3-5, 3-6, 3-7, 4-5, 4-6, 4-7, 5-6, or 5-7, or 7-10, 7-8, 7-9, 7-10, 8-9, 8-10, or 9-10. In certain aspects, the buffer of the second part is sodium acetate. The sodium acetate buffer can have a concentration of about 1 mM to about 500 mM such about 10, 20, 30, 40, 50, 60, 70, 80, 90, 100, 200, 300, 40 or about 500 mM.

In operation, the user will generally mix the two separate, previously prepared solutions; the first part containing the chemiluminescent substrate (e.g., luminol or a luminol derivative), phenol, optionally a co-enhancer and a stabilizer (i.e., the First Part) and the other part contains the oxidizing agent (i.e., the Second Part). The solutions should be appropriately buffered to maintain a working solution pH of about 6-12, preferably about 7-9.5 or 7-11. Suitable buffers include, but are not limited to, citrate, acetate, Tris [Tris(hydroxymethyl) amino methane], borate, carbonate and phosphate. In one aspect the buffer is a Tris buffer.

In general, the working solution is completely aqueous, although in order to achieve solubility of a particular enhancer, it may be necessary to include organic solvents such as dimethyl sulfoxide (DMSO) or alcohols. The working solution can generally be used at a temperature of about 10° C. to about 50° C., such as about 10, 15, 20, 25, 30, 35, 37, 40, 45, or 50° C.

In certain aspects, the formulation and preferably the first part includes one or more stabilizers. Suitable stabilizers include, but are not limited to, a Lewis acid, a tertiary amine, a urea, ascorbic acid, a thiol or a pyridine derivative. Advantageously, the stabilizer(s) enhance signal duration and shelf-life.

In one aspect, the stabilizer is a Lewis acid. Suitable Lewis acids include, but are not limited to, zinc acetate, zinc bromide, scandium triflate, sodium orthovanadate (Na₃VO₄) and boric acid. Those of skill in the art will know of other Lewis acids suitable for use in the present invention. In one aspect, the Lewis acid further comprises a tertiary amine (e.g., trialkylamine such as triethylamine) or a pyridine derivative. In certain aspects, the Lewis acid is present in the first part at about 0.01 mM to about 10 mM. In other aspects, the Lewis acid is present at about 0.1 to about 1.0 mM or 0.1, 0.2, 0.3, 0.4, 0.5, 0.6, 0.7, 0.8, 0.9, or about 1.0 mM. In one aspect, zinc acetate is present at about 0.4, 0.5, or 0.6 mM.

In one aspect, the stabilizer is a urea. Suitable ureas include, but are not limited to, dibenzylurea or diphenylurea or di(1H-imidazol-1-yl)methanone. The urea can be present from nanomolar (nM) quantities to micromolar (M) quantities. In certain instances, the urea is present from about 1 nM to about 10 μM. In other instances, the urea is present from about 1 nM, 10, 20, 30, 40, 50, 60, 70, 80, 90, 100, 200, 300, 400, 500, 600, 700, 800, 900 nm, 1 μM or 10 μM.

In certain aspects, the formulation or the urea formulation further comprises a tertiary amine or thiol. In certain aspects, the tertiary amine is present at about 0.01 mM to about 1.0 mM such as about or 0.1, 0.2, 0.3, 0.4, 0.5, 0.6, 0.7, 0.8, 0.9, or about 1.0 mM. Suitable tertiary amines include a trialkylamine or 4-dialkylaminopyridine.

In one aspect, the thiol is thiol urea or dithiothreitol. In certain instances, the thiol is present at about 0.01 μM to about 1.0 μM such as about 0.1, 0.2, 0.3, 0.4, 0.5, 0.6, 0.7, 0.8, 0.9, or about 1.0 μM.

In another aspect, the stabilizer is ascorbic acid. Ascorbate can be present from nanomolar (nM) quantities to micromolar (μM) quantities. For example, ascorbic acid can be present at 1 nM, 10, 20, 30, 40, 50, 60, 70, 80, 90, 100, 200, 300, 400, 500, 600, 00, 800, 900 nm, 1 μM or 10 μM. In certain instances, ascorbic acid is present at 0.1 μM to 1.0 μM such as 0.1, 0.2, 0.3, 0.4, 0.5, 0.6, 0.7, 0.8, 0.9, or about 1.0 μM.

In yet another aspect, the formulation or the urea formulation further comprises a co-enhancer such as a transition metal salt. Suitable transition metal salts include salts of Pd(0), Pd(II), Rh(III), Re(III), Ru(III), Ni(II), Mn(II), Mn(III) and combination thereof. In certain instances, the transition metal salt is present at about 0.1 μM to about 500 μM such about 0.1, 0.2, 0.3, 0.4, 0.5, 0.6, 0.7, 0.8, 0.9, 1, 1.5, 2, 3, 3.5, 4, 5, 6, 7, 7.5, 8, 9, 10, 20, 30, 40, 50, 60, 70, 80, 90, 100, 200, 300, 400 or about 500 μM.

In one aspect, the stabilizer is a pyridine derivative. Suitable pyridines derivative include, but are not limited to, 4-(dimethylamino)pyridine (DMAP), 4-morpholinopyridine (MORP) and 2-(9-1H-imidiazole-2-yl)pyridines (2-IP). In certain aspects, the pyridine derivative is present from micromolar (μM) quantities to millimolar (mM) quantities. In certain instances, the MORP is present from about 10 μM to about 5 mM. In other instances, the pyridine dervative is present from about 10 μM, 10, 20, 30, 40, 50, 60, 70, 80, 90, 100, 200, 300, 400, 500, 600, 700, 800, 900 μm, 1 mM, 2 mM, 3 mM, 4 mM, or 5 mM (1-5 mM).

In certain aspects, the formulation contains a detergent or other additive. Suitable additives include, Triton-X100, ethylene glycol, propylene glycol, and the like.

Peroxidase or luminol can be present in the form of a conjugated antibody tag used in an immunoassay to determine an antigen. Alternatively, peroxidase or luminol can be conjugated to a nucleotide, an oligonucleotide or a nucleic acid in hybridization assays. Therefore, the present invention is applicable to any method of diagnostic assay of a substance whose presence or amount is related to the presence or amount of a reactant such as luminol, a peroxidase enzyme, a catalyst, an oxidant, and/or a phenol enhancer that reacts in a chemiluminescent reaction, whose emission of light is detected or measured so that the presence or amount of material to be analyzed is related to the production of light. The present invention also includes a kit for performing an assay comprising luminol, an enhancer, and an oxidant. The kit optionally comprises instructions for use.

In one aspect, the chemiluminescent formulations, methods and systems of the present invention can be used in a wide variety of biological applications. These applications include, but are not limited to, an antibody-antigen reaction, an ELISA, a Western Blot, a Dot Blot, a Slot blot, a Southern Blot, or a Northern Blot or systems utilizing a labeled catalyst such as a peroxidase enzyme.

In one aspect, the present invention provides formulations and methods wherein an antibody-antigen reaction is carried out. For example, an antigen is allowed to react with a peroxidase-labeled antibody to form an antigen-antibody complex. The thus formed antigen-antibody complex is then allowed to react with a fixed antibody, so that the activity of the peroxidase is measured by chemiluminescence which is generated by the reaction using the formulation of the present invention e.g., between luminol or isoluminol serving as a substrate with a phenol enhancer in the presence of hydrogen peroxide. In operation, in order to enhance the duration and/or shelf-life of the emission of light of the substrate, a phenol and a stabilizer are employed, each with a concentration of about 5 to 300 μM, or with a concentration of about 10 to 200 μM. A co-enhancer is optionally employed.

The measurement of the light emission can be carried out after the previously mentioned antigen-antibody reaction by using a diacylhydrazide such as luminol or isoluminol with a concentration of 50 to 1000 μM, preferably with a concentration of 200 to 300 μM, and a hydrogen peroxide solution with a concentration of 100 to 1000 μM, in a buffer solution, for example, a tricine-NaOH buffer solution with pH 8 to pH 8.5.

In certain instances, the formulations, methods and systems of the present invention are used in Enzyme Linked Immunosorbent Assays (ELISAs), which utilize an enzyme label for the detection of proteins. Typically, a specific antibody is passively absorbed to a plate. The nonspecific sites are blocked with a protein solution which has no active part in the specific immunochemical reaction of a particular assay. A specific protein of interest is captured by the antibody on the surface and then detected by another antibody with an enzyme label. The enzyme label is reacted with a chemiluminescent formulation of the present invention and detected in a luminometer.

In a Western Blot application, a protein(s) is detected by first separating protein samples electrophoretically on for example, a SDS polyacrylamide gel. The proteins are then transferred electrophoretically to a membrane such as nitrocellulose. The nonspecific sites are blocked with a protein solution that has no active part in the specific immunochemical reaction of a particular assay. A specific protein of interest is detected then by the addition of an antibody made against the protein. After a wash step to remove any unbound antibody, a peroxidase labeled antibody is added that will react with the primary antibody. The unbound enzyme labeled antibody is removed by a series of wash steps. The membrane is then exposed to the chemiluminescent formulation of the present invention to produce light. The membrane is then exposed to film or other detection medium for detection.

In a Dot Blot, proteins are directly applied to a membrane and detected with a previously described antibody system.

In a Southern blot, DNA is detected by first separating the DNA sample electrophoretically on an agarose gel. The DNA is then transferred to a membrane such as charge-modified nylon. The DNA is then fixed by irradiation or baking. The membrane is then blocked with a prehybridization buffer to prevent any nonspecific binding of a DNA probe. The DNA probe coupled to a detectable label such as biotin is then added to the membrane and is allowed to incubate for several hours at 50° C. or higher. The blots then undergo a series of stringency washes to remove any nonspecific hybridized probe from the DNA target while maximizing target/probe interactions. The blots are blocked again to prevent any nonspecific binding of the enzyme labeled probe. A peroxidase labeled conjugate such as streptavidin peroxidase is added to the membrane. The membrane is washed to remove any unbound label. The membrane is then exposed to the chemiluminescent substrate to produce light. The membrane is then exposed to film or other detection medium for detection.

In a Northern blot, RNA is detected by separating RNA samples and detecting with a DNA or RNA probe using a method similar to the Southern Blot application. Care must be taken to remove all ribonucleases which can interfere and destroy the target.

The formulations and methods provide a high degree of luminescence, which is develops rapidly. The intense luminescence persists for a longer period of time compared to other phenol based systems. Thus, by using the methods of the present invention, rapid development of high intensity luminescence is achieved and the luminescence is of an extended duration. Moreover, by combining the features of high light output with extended duration, unprecedented levels of sensitivity are achieved in many assay systems.

In certain aspects, the present invention provides methods using a catalytic reagent that functions similarly like a peroxidase, such as horseradish peroxidase (HRP), or cytochrome C, but is not an enzyme. The catalytic agent is a phthalocyanine metal catalyst and is disclosed in U.S. patent application Ser. No. 14/262,659, filed Apr. 25, 2014, the disclosure of which is hereby incorporated by reference.

In another embodiment, the present invention provide a kit for chemiluminescence, the kit comprising, consisting essentially of, or consisting of a first part and a second part:
a) the first part comprising:
   i) a diacylhydrazide selected from the group consisting of luminol, isoluminol or a luminol derivative;
   ii) a phenol enhancer;
   iii) optionally one or more co-enhancers;
   iv) one or more stabilizers; and
b) the second part comprising:
   v) an oxidant, in the presence of a peroxidase to provide light emission from a chemiluminescence formulation and instructions for use.

In operation, the user will generally mix the two separate, previously prepared solutions; the first part containing the chemiluminescent substrate (e.g., luminol or a luminol derivative), phenol and a stabilizer (i.e., the First Part) and the other part contains the oxidizing agent (i.e., the Second Part). The solutions should be appropriately buffered to maintain a working solution pH of about 6-12, preferably about 7-9.5.

### III. Examples

### Example 1

### Luminol Substrate Containing a Phenol Enhancer

This example illustrates the use of a phenol enhancer, e.g., 4′-hydroxy-4-biphenylcarboxylic acid (BIPCA) in a luminol based substrate for horseradish peroxidase (HRP). Typically, chemiluminescent substrates comprising a luminol solution and a stable peroxide solution are mixed together to form the substrate prior to use. In this example, the performance of the BIPCA substrate solution on Western blots was assessed in comparison to the SuperSignal® West Dura substrate (Pierce Biotechnology, Rockford, Ill.).

The BIPCA substrate solution was made by mixing equal volumes of the luminol solution (solution A) containing 200 mM Tris, 0.6 mM BIPCA and 7 mM luminol, and the peroxide solution (solution B) containing 10 mM sodium perborate and 50 mM sodium acetate. The substrate solution was incubated for 5 minutes at room temperature with blots probed with an antibody labeled with HRP before image acquisition.

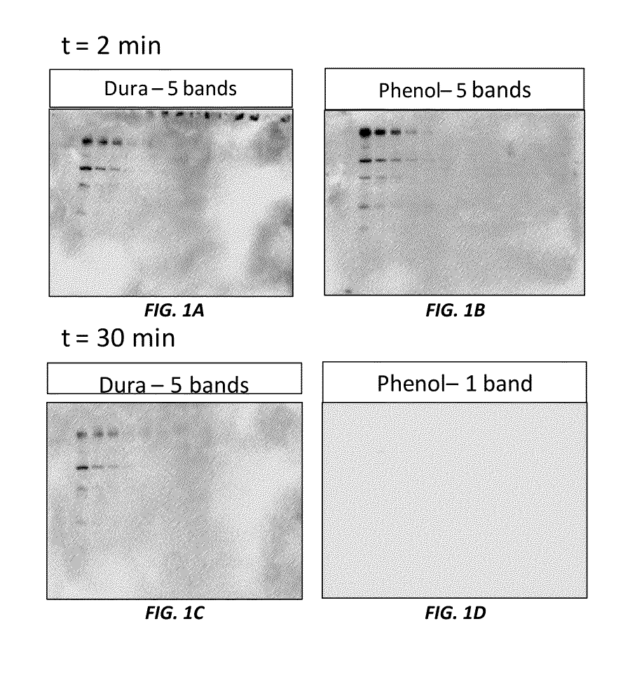

The BIPCA substrate solution showed high signal sensitivity at the early acquisition time point (e.g., 2 minutes). See, FIG. 1B. The signal was comparable or slightly superior to that of the Dura substrate (FIG. 1A). However, at the later time point (e.g., 30 minutes) the signal from the BIPCA substrate was almost undetectable (FIG. 1D). This was in contrast to the signal from the Dura substrate (FIG. 1C).

This example shows that the BIPCA substrate solution has high sensitivity and high intensity at initial time points during image acquisition, but has a short signal duration (e.g., <30 minutes). The data shows that the signal intensity was high after 2 minutes and was greatly reduced at 30 minutes.

To demonstrate the stabilizing effect, stabilizers of the present invention were included. In a 96 well-plate were added 50 μL Solution a (Part 1) (120 nM urea, 250 μM DMAP, 10.5 mM luminol, 0.6 mM BIPCA, 3% ethylene glycol, 0.03% Triton X-100, 300 mM Tris), 50 μL. Solution B (Part 2) (25 mM sodium acetate and 5 mM sodium perborate solution in water) was added followed by 5 μl of HRP solution (1/1000 of 1 mg/mL solution). The plate was then placed inside a BioTek plate reader and the chemiluminescence was measured with time in 10 min interval. The signal duration percentage significantly decreased to 37%.

### Example 2

### Luminol Substrates Containing Phenol Enhancers in Combination Lewis Acid Catalysts and Tertiary Amine Stabilizers

This example shows a comparison of luminol substrates that contain a phenol enhancer as well as a Lewis acid catalyst and a tertiary amine. Lewis acid catalysts such as scandium(III) triflate and zinc triflate, and triethylamine were added to the BIPCA enhancer described above in an effort to prevent signal decay. The results indicate that the Lewis acid catalyzed Michael addition quenched the quinine produced during oxidation of the phenol enhancer.

A series of substrate formulations (e.g., Formulations #27-30) were made and their performance was compared to commercially available chemiluminescent substrates, such as the SuperSignal® West Dura and the SuperSignal® West Pico substrates (Pierce Biotechnology). The luminol solution of the test substrates were formulated as follows: #27 contained 200 mM Tris, 7 mM luminol, 0.3 mM BIPCA, 0.6 mM Zn Ac, and 0.6 mM triethylamine; #28 included 200 mM Tris, 7 mM luminol, 0.6 mM BIPCA, 0.3 mM Zn Ac, and 0.3 mM triethylamine; #29 contained 200 mM Tris, 7 mM luminol, 0.6 mM BIPCA, 0.3 mM scandium triflate, and 0.3 mM Zn Ac; and #30 contained 200 mM Tris, 7 mM luminol, 0.3 M BIPCA, 0.15 mM scandium triflate, and 0.15 mM triethylamine. The substrates were tested on Western blots to detect ERK2 in NIH/3T3 cell lysates. A serial dilution of the cell lysate (10 μg to about 1.2 ng of protein) was run on a polyacrylamide gel and blotted onto a nitrocellulose membrane according to standard Western blotting methods. The membrane was blocked with 5% milk, probed with an anti-ERK2 mouse monoclonal antibody (Cat. No. sc-1647, Santa Cruz Biotechnology, Santa Cruz, Calif.), and incubated with a secondary goat anti-mouse antibody conjugated to HRP (Cat. No. 111-035-146, Jackson ImmunoResearch Laboratories, West Grove, Pa.). To form the test substrate solutions, the luminol solutions described above were mixed in an equal volume with the peroxide solution containing 10 mM sodium perborate and 50 mM sodium acetate. The resulting test substrates were incubated with the blots for 5 minutes at room temperature prior to imaging on the Odyssey® Fc system (LI-COR, Lincoln Nebr.). Images were taken at time 0, 30 minutes and 1 hour during the acquisition period.

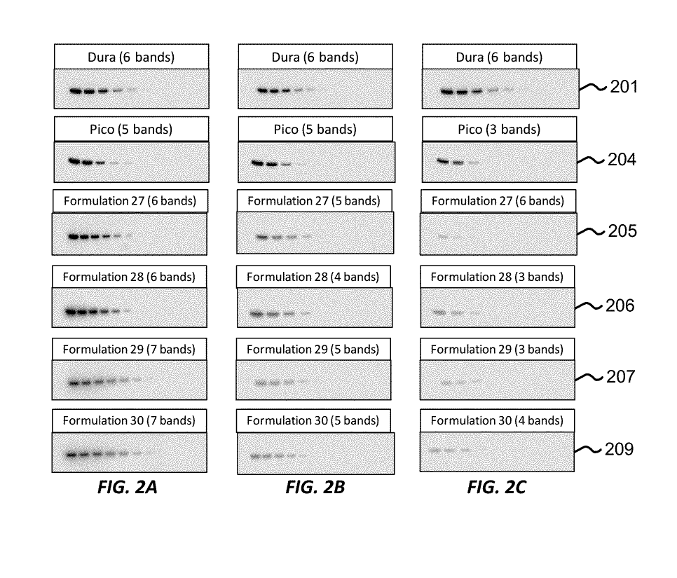

Images of the blots at the various acquisition time points are provided in FIGS. 2A-C. The blots at the 2 minute time point are shown in FIG. 2A. The signal intensity of substrates #27-30 was similar to that of the Dura and Pico substrates. The signal sensitivity was better (e.g., higher) with substrates #29-30 compared to the Dura and Pico substrates. For example, 7 bands were detected in the blots incubated with these formulations and 6 bands were detected with Dura. At the 30 minute time point (FIG. 2B), the signal intensity and sensitivity was decreased for substrates #27-30 compared to the controls and the earlier time point. Similarly, at the 60 minute time point (FIG. 2C), the signal intensity and sensitivity was lower for substrates #27-30 (205, 206, 207, 209) compared to Dura (201) and Pico (204). The signal from the Dura substrate remained the same across the time points. The test substrates showed signal decay while the control substrates did not.

### Example 3

### Luminol Substrates with Stabilizers for Quenching Quinones

This example shows a comparison of luminol substrates containing phenol enhancers and various co-enhancers including (a) a Lewis acid catalyst and a tertiary amine, (b) urea, and (c) ascorbic acid. These stabilizers were used to quench quinones formed during phenol oxidation of the substrate.

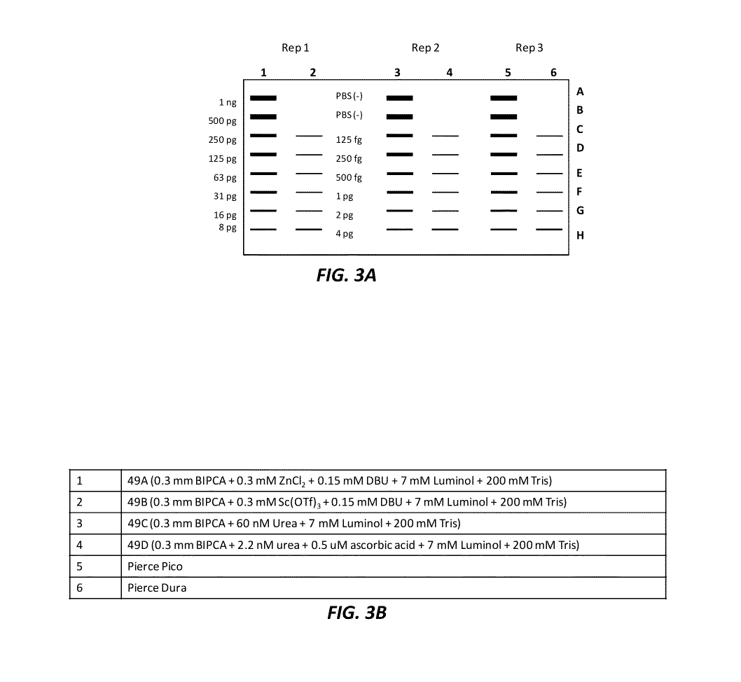

The luminol solution of the test substrates were formulated as follows (FIG. 3B): #49A contained 0.3 mM BIPCA, 0.3 mM ZnCl₂, 0.15 M DBU, 7 mM luminol, and 200 mM Tris; #49B contained 0.3 mM BIPCA, 0.3 mM Sc(OTf)₃, 0.15 M DBU, 7 mM luminol, and 200 mM Tris; #49C contained 0.3 mM BIPCA, 60 nM urea, 7 mM luminol, and 200 mM Tris; #49D contained 0.3 mM BIPCA, 2.2 nM urea, 0.5 μM ascorbic acid, 7 mM luminol, and 200 mM Tris. The test substrate solutions were made by mixing the luminol solutions with an equal volume of the peroxide solution containing 10 mM sodium perborate and 50 mM sodium acetate. The test substrates were compared to Dura and Pico substrates (Pierce Biotechnology).

The substrates were tested on slot blots of an HRP labeled goat anti-mouse antibody. A serial dilution of the antibody from 1 ng to 125 fg was spotted on an Odyssey® nitrocellulose membrane in triplicate. The blots were blocked with 5% skim milk for 1 hour and incubated for 5 minutes with the test substrates. Images were acquired on an Odyssey® Fc at 2 minute exposures at an acquition time point of 0 and 1 hour.

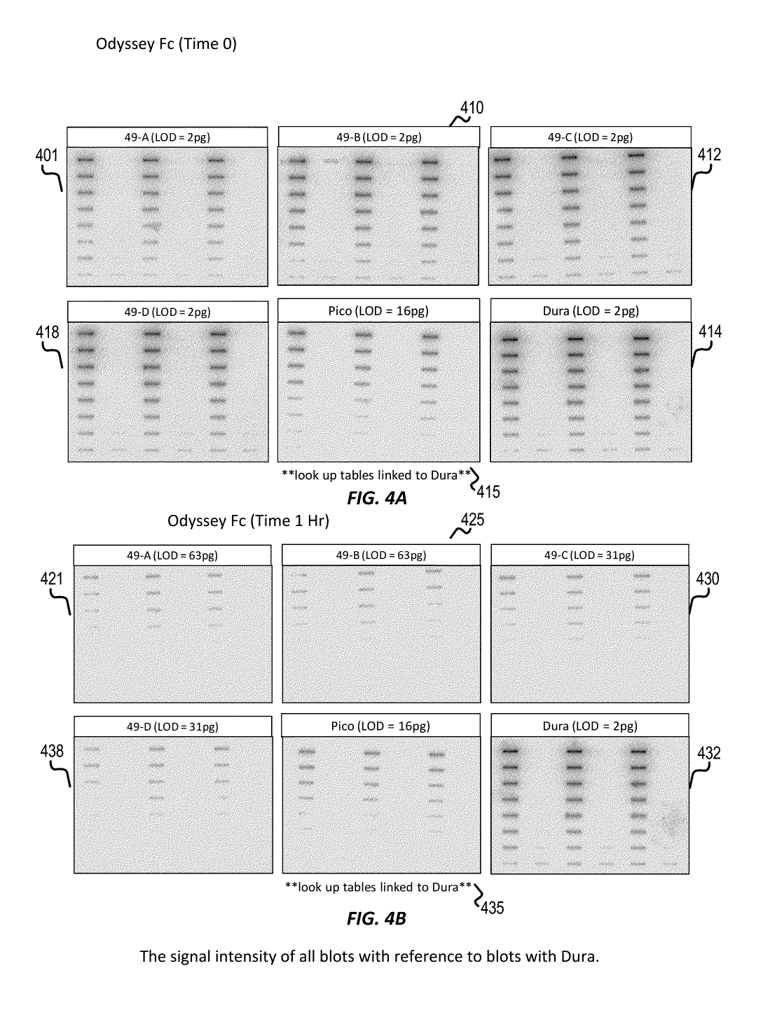

FIG. 4A shows images of the blots at time 0. All the test formulations (#49A-49D, 401, 410, 412, 418) gave similar results as the Dura (414) and Pico (415) substrates at the initial time point. The lower limit of detection (LOD) for the test formulations was the same (e.g., 2 pg) as the Dura substrate and lower than the Pico substrate (e.g., 16 pg). At the later time point of 1 hour (FIG. 4B), the signal intensity and sensitivity of the test substrates (421, 425, 430, and 438) was reduced compared to the Dura (432) and Pico (435) substrates. The test substrates showed reduced signal duration. For instance, the signal intensity was decreased at time 1 hour compared to time 0. The LOD also changed from 2 pg at time 0 (401 and 418) to 31 pg (438) or 63 pg (421) one hour later.

### Example 4

### Luminol Substrates Containing Phenol in Combination with Urea and Ascorbic Acid Stabilizers

This example illustrates the use of BIPCA based luminol solutions that contain urea and acetic acid for chemoiluminescence with horseradish peroxidase (HRP). In this study, the performance of the BIPCA substrate solutions were compared to SuperSignal® West Dura substrate (Pierce Biotechnology) on slot blots.

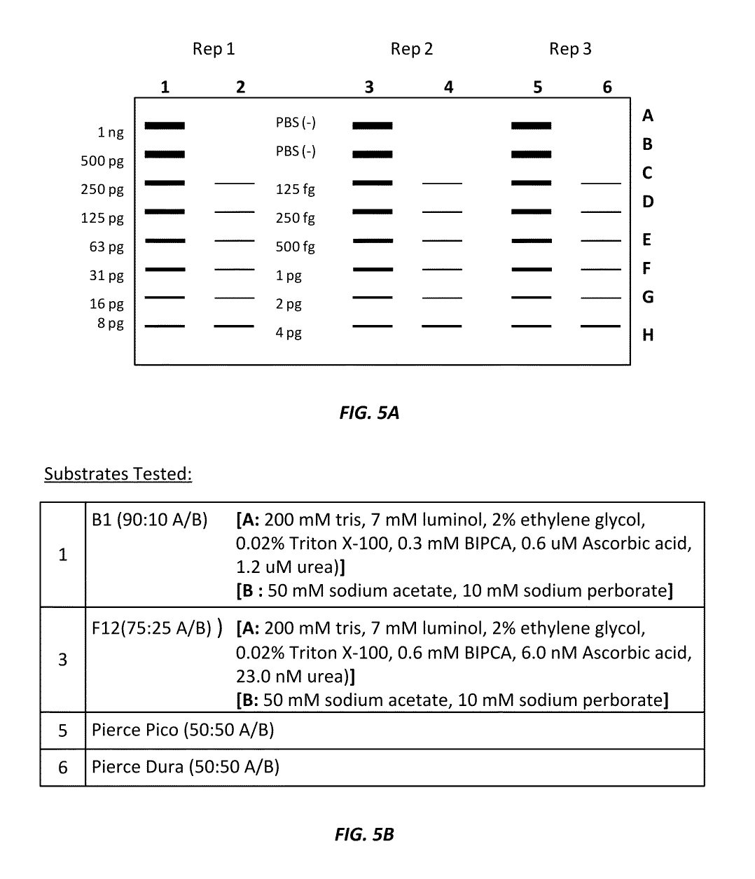

Two test substrates (B1 and F12) were made as follows (FIG. 5B). For the B1 substrate, the luminol solution and peroxide solution were mixed at a ratio of 90:10. The B1 luminol solution contained 200 mM Tris, 7 mM luminol, 2% ethylene glycol, 0.02% Triton X-100, 0.3 mM BIPCA, 0.6 μM ascorbic acid, and 1.2 μM urea. The B1 peroxide solution contained 50 mM sodium acetate and 10 mM sodium perborate. For the F12 substrate, the luminol solution and peroxide solution were mixed at a ratio of 75:25. The F12 luminol solution contained 200 mM Tris, 7 mM luminol, 2% ethylene glycol, 0.02% Triton X-100, 0.6 mM BIPCA, 60 nM ascorbic acid, and 23 nM urea. The peroxide solution for both substrates included 50 mM sodium acetate and 10 mM sodium perborate. When indicated, look-up tables were used to adjust the appearance of the image. The look-up tables display histograms of the pixel intensities for each channel acquired for the image. For reference, when stated the look-up tables are linked, this means the display histograms are identical for each channel.

The substrates were tested on slot blots of an HRP labeled goat anti-mouse antibody (FIG. 5A). A serial dilution of the antibody from 1 ng to 125 fg was spotted on an Odyssey® nitrocellulose membrane in triplicate. The blots were blocked with 5% skim milk for 1 hour and incubated for 5 minutes with the test substrates. Images were acquired on an Odyssey® Fc at 2 minute exposures at an acquition time point of 0 and at 30 minutes.

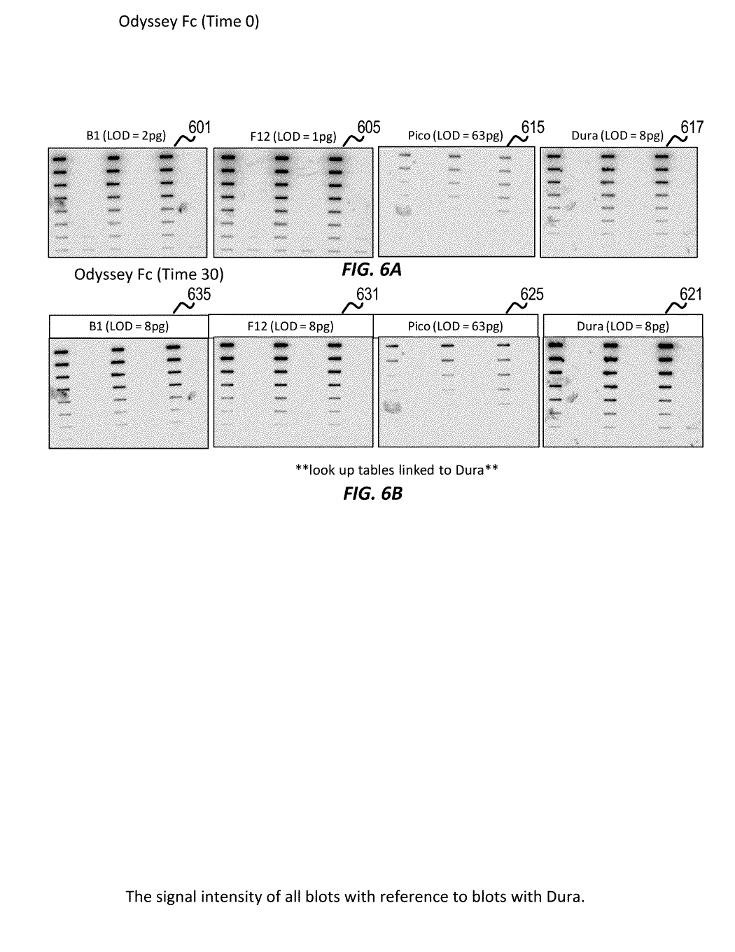

FIG. 6A shows images of the blots at acquition time 0. The B1 (601) and F12 (605) substrates had greater signal intensity and sensitivity than both the Dura (617) and Pico (615) substrates. The lower limit of detection (LOD) for the test substrates was 2 pg for B1 and 1 pg for F12. In contrast, the LOD for the control substrates was higher at 8 pg for Dura and 63 pg for Pico. At time 30 minutes (FIG. 6B), the B1 (635) and F12 (631) substrates produced a robust signal that was similar to the signal from the Dura substrate (621) and stronger than the one from the Pico substrate (625).

The results show that increasing the Tris, luminol and BIPCA concentrations improved the signal intensity and signal duration compared to the test substrates described in Examples 1 and 2. Urea and ascorbic acid also improved the signal intensity and signal duration.

### Example 5

### Performance of Phenol Enhancer with Urea, Tertiary Amine and Thiol Stabilizers

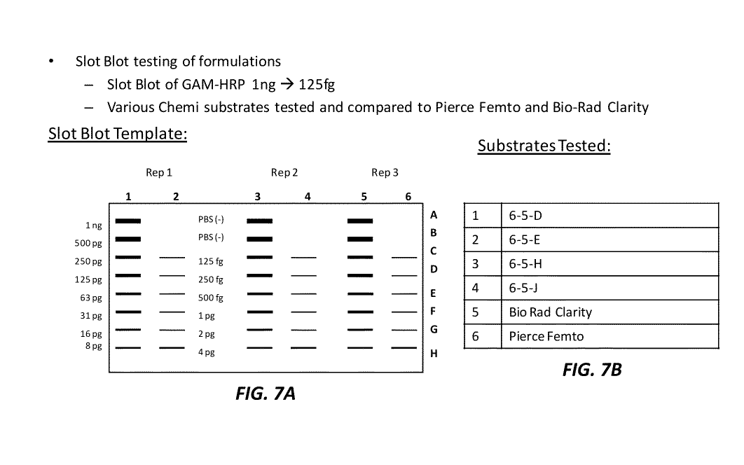

This example shows the activity of chemiluminescent substrates containing a phenol enhancer based luminol solution and a peroxide solution with 5 mM sodium perborate and 25 mM sodium acetate. The BIPCA based luminol solutions tested were as follows: #6-5-D contained 10.5 mM luminol, 0.6 mM BIPCA, 3% ethylene glycol, 0.03% Triton X-100, 300 mM Tris, 120 nM urea, and 250 μM 4-dimethylaminopyridine (DMAP); #6-5-E contained 10.5 mM luminol, 0.6 mM BIPCA, 3% ethylene glycol, 0.03% Triton X-100, 300 mM Tris, 120 nM urea, and 0.5 μM DTT; #6-5-H contained 10.5 mM luminol, 0.6 mM BIPCA, 3% ethylene glycol, 0.03% Triton X-100, 300 mM Tris, 120 nM urea, and 120 nM thiourea; and #6-5-J contained 10.5 mM luminol, 0.6 mM BIPCA, 3% ethylene glycol, 0.03% Triton X-100, 300 mM Tris, 23 nM urea, and 48 μM ascorbate. In this study, the performance of the co-enhancer substrate solutions were compared to Bio-Rad's Clarity™ Western ECL substrate and Pierce's SuperSignal® West Femto substrate on slot blots (FIG. 7B).

The substrates were tested on slot blots of an HRP labeled goat anti-mouse antibody (FIG. 7A). A serial dilution of the antibody from 1 ng to 125 fg was spotted on an Odyssey® nitrocellulose membrane in triplicate. The blots were blocked with 5% skim milk for 1 hour and incubated for 5 minutes with the test substrates. Images were acquired on an Odyssey® Fc at 2 minute exposures at an acquition time point of 0 and at 1 hour.

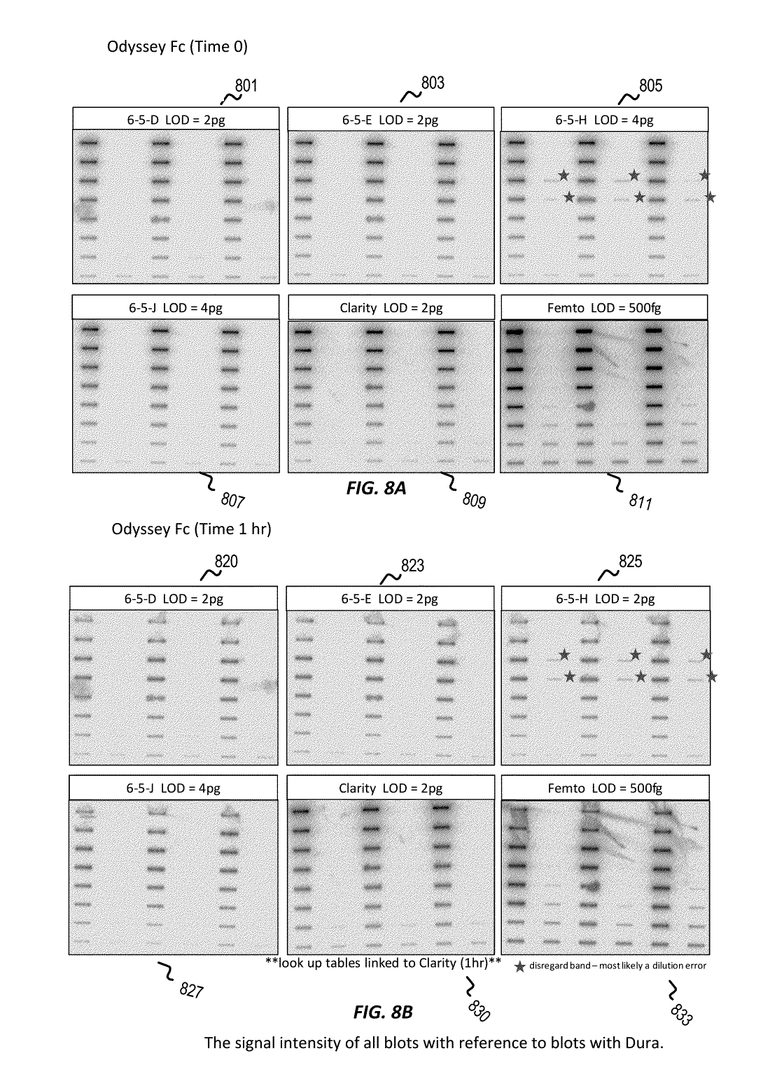

FIG. 8A shows images of the blots at time 0. The signal intensity and sensitivity of the test substrates (801, 803, 805 and 807) was similar to the Clarity™ substrate (809). In addition, the lower limit of detection was also the same for these substrates. The Femto substrate had a lower LOD of 500 fg (811). At time 1 hour, the test substrates showed signal stability (FIG. 8B; 820, 823, 825, 827). Their signal duration was similar to the Clarity™ substrate (830) and the Femto substrate (833). The signal from the test substrates was also detectable at time 2 hours (data not shown).

The data shows that increasing Tris, luminol, and BIPCA concentration while decreasing sodium perborate and sodium acetate concentration improved the signal intensity and signal duration. In addition, urea and urea derivatives along with DMAP, DTT and ascorbic acid also improved the performance of the substrate.

### Example 6

### Phenol-Based Chemiluminescent Substrate with Increased Signal Duration

This example shows the performance of an exemplary embodiment of the present invention. The chemiluminescent substrate formulation includes a luminol solution containing 350 mM Tris, 10.5 mM luminol, 0.8 mM BIPCA, 1 μM urea, 1 mM DMPA, 0.03% Triton X-100, and 2.0% ethylene glycol, and a peroxide solution of 5 mM sodium perborate and 25 mM sodium acetate.

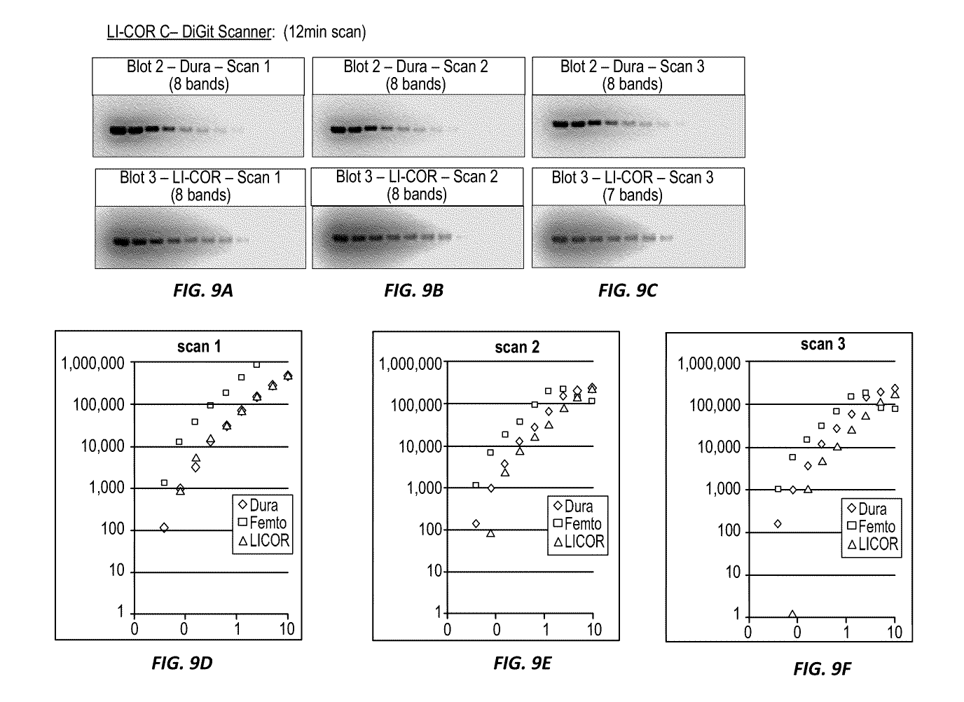

The substrate was tested on a Western blot to detect actin in C32 cell lysates. A serial dilution of the cell lysate (10 μs to about 1.2 ng of protein) was run on a polyacrylamide gel and blotted onto a nitrocellulose membrane according to standard Western blotting methods. The membrane was blocked with 5% milk, probed with an anti-actin mouse monoclonal antibody and incubated with a secondary goat anti-mouse antibody conjugated to HRP. The test substrate was incubated with the blot for 5 minutes at room temperature prior to imaging on the C-DiGit® blot scanner (LI-COR). Three 12-minute scans were performed back-to-back (FIGS. 9A-C). The images were not linked and were individually optimized. Pierce's Dura substrate served as the control substrate.

The chemiluminescent substrate formulation performed similarly to Pierce's Dura substrate (FIGS. 9D-F). The formulation showed slightly less signal intensity compared to Pierce's Femto substrate. The signal stability of the test substrate was the same as the control substrates.

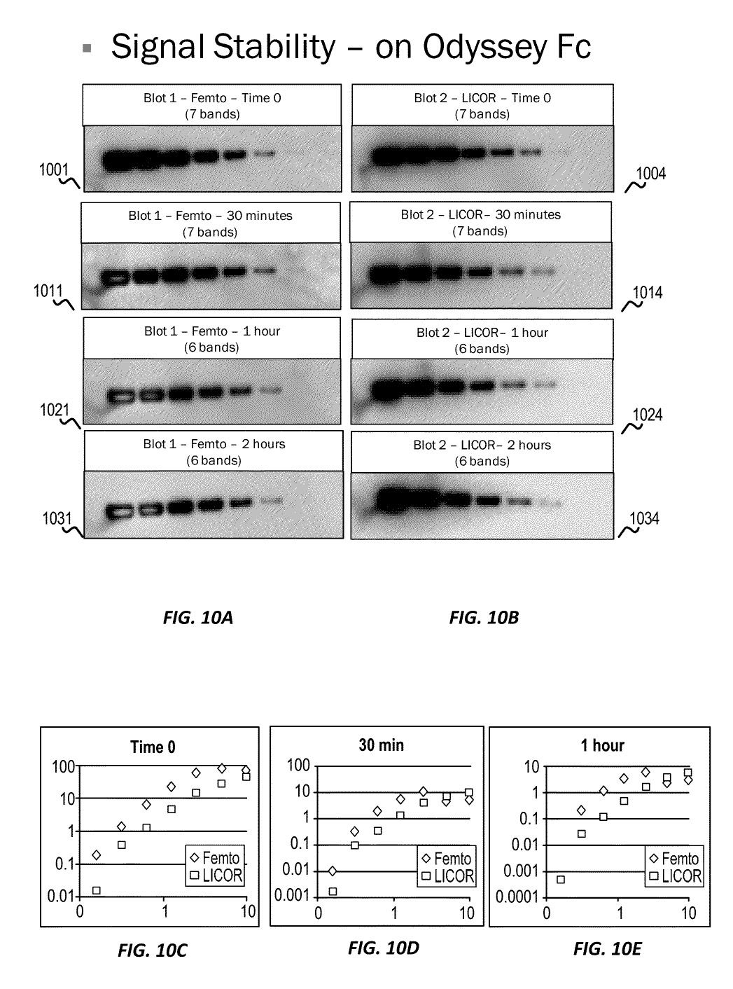

Similar Western blots were imaged on the Odyssey® Fc with a 2 minute acquisition window at time 0, 30 minutes, 1 hour and 2 hours. FIGS. 10A-B show a comparison between the control substrate (Pierce's Femto substrate) and the test substrate (1001 vs. 1004; 1011 vs. 1014; 1021 vs. 1024; 1031 vs. 1034). Both substrates showed similar signal stability across the time points. 7 bands were detected at time 0 and 6 bands were visible at time 2 hours using either substrate. Quantitative data shows that the test and Femto substrates performed similarly at time 0 (FIG. 10C), 30 minutes (FIG. 10D) and 1 hour (FIG. 10E).

The substrate was also tested on a Western blot to detect ERK2 in NIH/3T3 cell lysates. A serial dilution of the cell lysate (2.5 μg to about 39 ng of protein) was run on a polyacrylamide gel and blotted onto a nitrocellulose membrane according to standard Western blotting methods. The membrane was blocked with 5% milk, probed with an anti-actin mouse monoclonal antibody, and incubated with a secondary goat anti-mouse antibody conjugated to HRP. The test substrate was incubated with the blot for 5 minutes at room temperature prior to imaging on the Odyssey® Fc (LI-COR). The images were not linked and were individually optimized. The control substrates used were Pierce's Femto, Dura and Pico substrates.

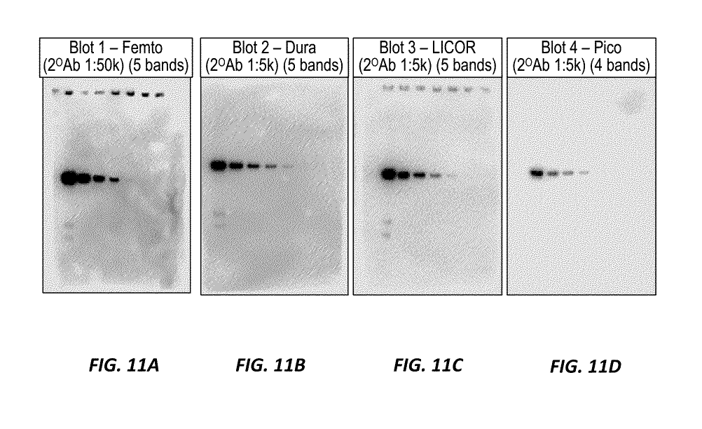

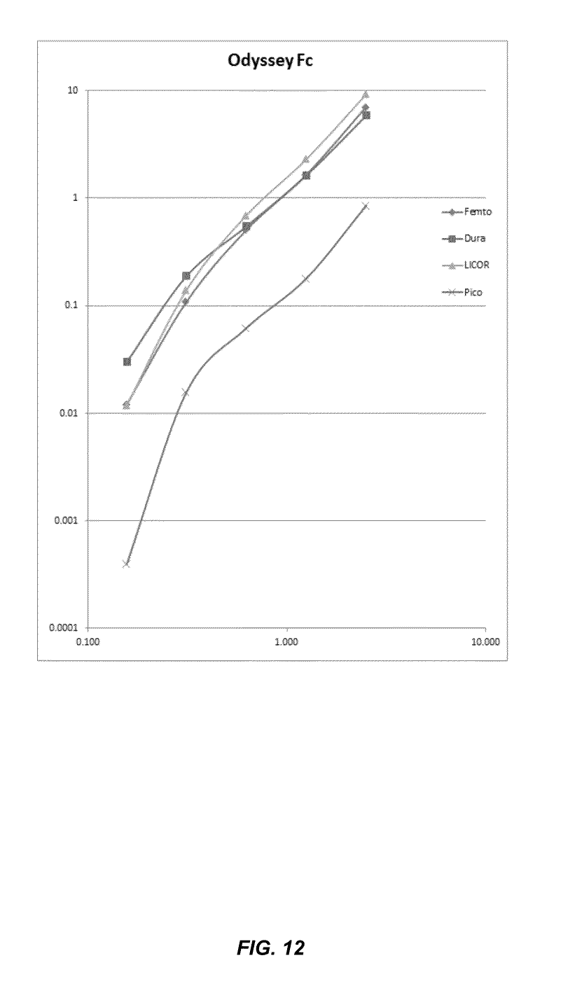

The images of the blots (FIGS. 11A-D) and the quantitative data (FIG. 12) show that the test substrate performed as well as the control substrates Dura and Femto.

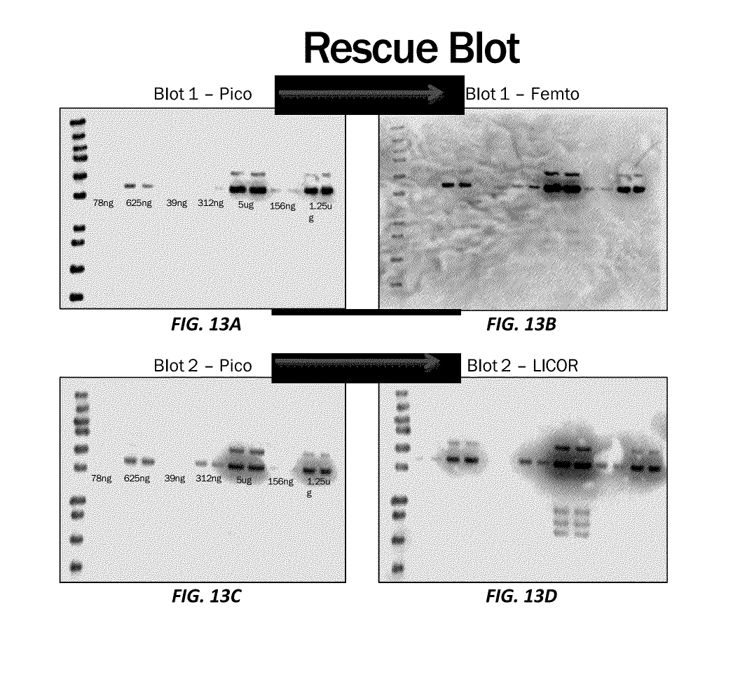

Many times a lower cost substrate is used initially with a Western Blot, however if the protein band of interest is not visualized, a more sensitive substrate i.e. Femto is used on the same blot. This is often termed as a “rescue blot.” The activity of the test substrate on rescue blots was also assessed. Western blots were first incubated with the Pico substrate (FIGS. 13A and 13C) and then incubated with either the Femto substrate (FIG. 13B) or the test substrate (FIG. 13D). The test substrate had higher signal sensitivity compared to the control commerically available substrates.

The results demonstrate that the test substrate can be used in Western blotting for highly sensitive detection of proteins. The substrate's signal sensitivity was comparable to Pierce's Dura substrate. The test substrate was compatible with different imager systems, e.g., LI-COR's C-DiGit® and Odyssey® Fc imagers and standard autoradiograph films.

### Example 7

### Phenol-Based Chemiluminescent Substrate Containing a Transition Metal Complex

This example shows the performance of an exemplary embodiment of the present invention. A chemiluminescent substrate formulation containing a phenol enhancer, DMAP, urea and a transition metal complex was tested on a Western blot. The activity of the substrate was compared to a control substrate, e.g., SuperSignaly West Femto substrate (Pierce Biotechnology) as well as the test formulation without the transition metal complex.

The test substrate formulation included a luminol solution containing 350 mM Tris, 10.5 mM luminol, 0.8 mM BIPCA, 1 μM urea, 1 mM DMPA, 0.03% Triton X-100, and 2.0% ethylene glycol, and 160 μM water soluble palladium (Pd(0)) and a peroxide solution of 5 mM sodium perborate and 25 mM sodium acetate.

The test substrate was tested on a Western blot to detect actin in C32 cell lysates. A serial dilution of the cell lysate (10 μg to 156 ng of protein) was run on a polyacrylamide gel and blotted onto a nitrocellulose membrane according to standard Western blotting methods. The membrane was blocked with 5% milk, probed with an anti-actin mouse monoclonal antibody and incubated with a secondary goat anti-mouse antibody conjugated to HRP. The test substrate was incubated with the blot for 5 minutes at room temperature prior to imaging on the Odyssey® Fc. Images were acquired at multiple time points such as at time 0, 15 minutes, 30 minutes, 1 hour and 2 hours.

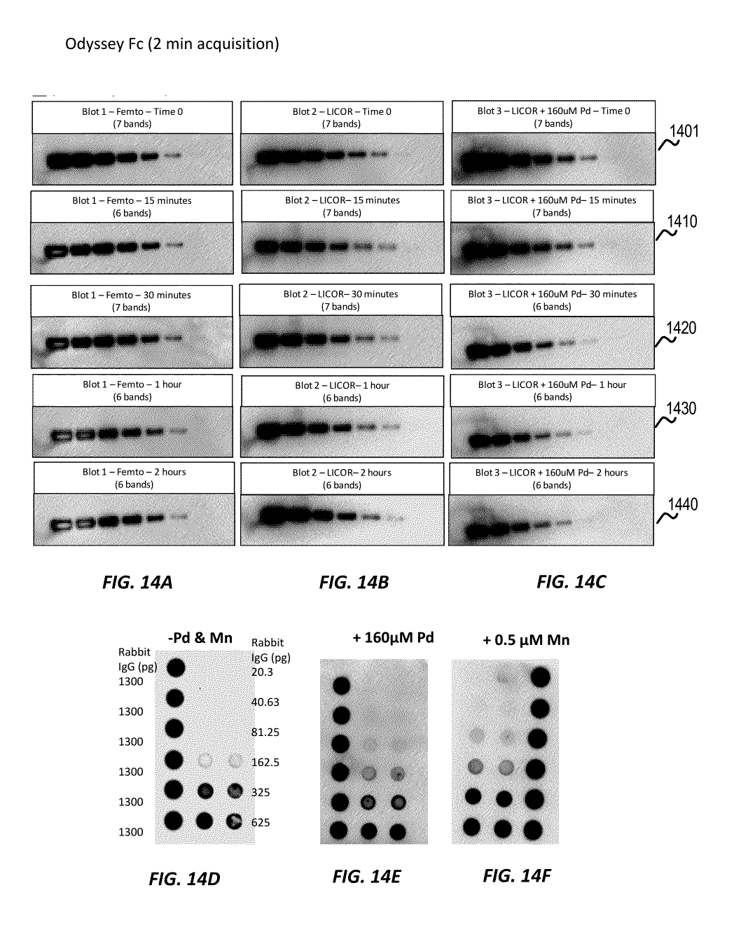

FIG. 14A shows the images of the blot treated with the Femto substrate at the different acquisition time points. FIG. 14B shows the images of the blot treated with the phenol enhancer substrate that does not contain transition metal complexes. FIG. 14C shows the images of the blot treated with the test substrate containing the phenol enhancer and palladium. The test substrate reached the same sensitivity observed for the most sensitive chemiluminescence Western blot substrate that is commercially available (SuperSignal® West Femto substrate, Pierce Biotechnology). The test substrate showed a high level of signal intensity and signal sensitivity at time 0 (1401), 15 minutes (1410), 30 minutes (1420), 1 hour (1430) and 2 hours (1440). The duration of the signal was the same for the Femto substrate and the test substrate containing palladium.

Transitional metal complexes were tested in the formulation to enhance sensitivity of the peroxidase assay. Solution A contains a water soluble Pd complex or a Mn(II) complex or a combination of both. Substrate performance was evaluated by the detection of Rabbit IgG on a dot blot. The formulation without a translational metal complex was used as a comparison. The working solution contains 7.5 mM Luminol, 0.4 mM BIPCA, 200 mM Tris, 100 mM Boric acid, 0.5 mM MORP, 2% ethylene glycol (EG), 2 mM H₂O₂ with (i) 160 μM Pd complex or (ii) 0.5 Mn(II) complex. As shown in FIG. 14E-F, these two transitional metal complexes are co-enhancers for chemiluminescence substrates.

### Example 8

### Luminol Substrates Containing Phenol in Combination with Urea and Tertiary Amine Stabilizers

In a 96 well-plate were added 50 μL solution containing 0.6 mM Coumaric acid, 300 mM tris base, 7 mM luminol sodium, 0.02% triton X-100, 2% ethylene glycol (“EG”) with stabilizers in water (120 nM urea or 200 μM DMAP). A control for this experiment is the same phenol formulation with out the stabilizers (0.6 mM Coumaric acid, 300 mM tris base, 7 mM luminol sodium, 0.02% triton X-100, 2% EG). Solution B (50 mM sodium acetate and 10 mM sodium perborate solution in water) is added to the control and test formulation followed by 5 μL of HRP solution (1/1000 of 1 mg/mL solution). The plate was then placed inside a BioTek plate reader and the chemiluminescence was measured with time in 10 min interval. Results show the addition of the stabilizers urea and DMAP increase the signal duration as seen in the signal duration percentage drop decreasing.

### Example 9

### Luminol Substrates Containing Phenol in Combination with Urea as a Stabilizer

In a 96 well-plate were added 504 Solution A (Part 1) (A5=10.5 mM Luminol, 3% EG, 0.03% Triton X-100, 300 mM Tris, 120 nM Urea, 0.6 mM, 4-(1,2,4-Triazol-1-yl)phenol, 2-(1H-Imidazol-2-yl)pyridine (“4-IP”), A6=10.5 mM Luminol, 3% EG, 0.03% Triton X-100, 300 mM Tris 120 nM Urea 0.6 mM BIPCA) with Dura as a reference, Solution B (part 2) (25 mM sodium acetate and 5 mM sodium perborate solution in water) was added followed by 5 uL of HRP solution (1/1000 of 1 mg/mL solution). The plate was then placed inside a BioTek plate reader and the chemiluminescence was measured with time in 10 min interval. The control for this experiment is the removal of the stabilizers. Control-A5=10.5 mM Luminol, 3% EG, 0.03% Triton X-100, 300 mM Tris, 0.6 mM 4-IP; Control A6=10.5 mM Luminol, 3% EG, 0.03% Triton X-100, 300 mM Tris 0.6 mM BIPCA). Results show that for A5, the signal duration dropped only 38.7%, however the intensity was not as high as the A6 formula. The signal duration of this formulation dropped significantly more than the A5 and Dura reference at 56%. Compared to the control, the signal duration percentage drop is significantly less with a stabilizer present.

### Example 10

### A High pH Buffer for the Second Part

A rabbit IgG dot blot is used for testing substrate performance. Bottle A solution is made from 15 mM Luminol, 0.8 mM BIPCA, 400 mM Tris, 200 mM Boric acid, 1 mM Na₃VO₄, and 2% EG (pH 10.4). Bottle B solution comprises 4 mM NaBO₃ in 0.1M Tris buffer (pH 8.5). The components in bottle B can also be made from 4 mM H₂O₂ in 0.1M 2-methyl-2-amine-1-propanol buffer (pH 9.8).

The working solution is prepared by mixing equal parts of bottle A with bottle B. A chemiluminescent signal is detected after incubation with the substrate with HRP-GAR (1 to 35K dilution) bound Rabbit IgG.

### Example 11

### Boric Acid and Sodium Orthovanadate as Chemi-Signal Stabilizers

The formulated substrates were tested on a dot-blot. A series of double dilution of Rabbit IgG from 1.3 ng to 83.2 pg was spotted on a nitrocellulose membrane. The Rabbit IgG was probed by HRP-GAR (1 to 35K dilution) and detected by four formulated chemiluminescent substrates. To ensure long-term stability, two solution were prepared. The working solution was obtained by mixing equal volumes of the two solutions of the following formulations:

Solution A: 10 mM Luminol, 0.8 mM BiCPA, 400 mM Tris, with a various of 50 mM, 100 mM and 200 mM Boric acid (pH 10.72): Solution B: 8 mM NaBO₃ in 50 mM sodium acetate (pH 5.1).

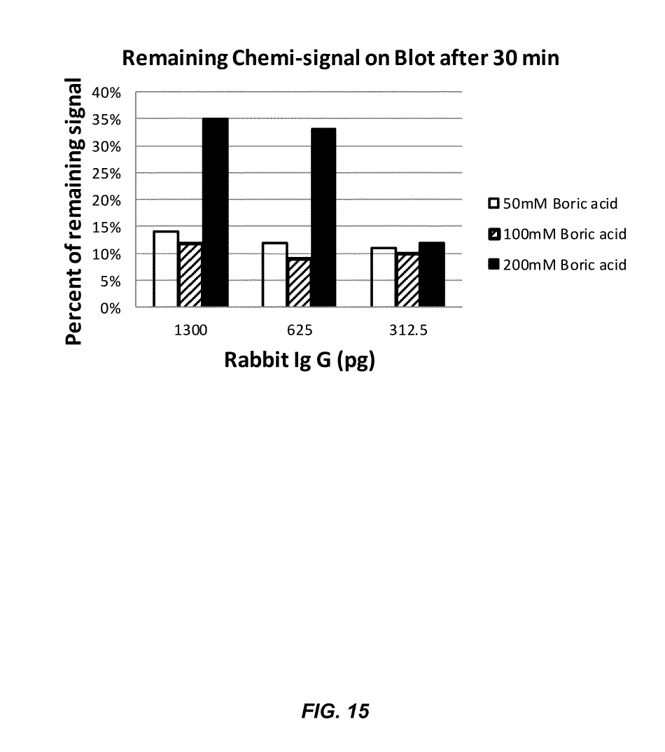

The blot was incubated with a substrate working solution for 5 minutes and then stored in a plastic sheet. The signal was recorded (defined as time 0) and after 30 minutes. FIG. 15 shows the remaining signal for detection of Rabbit IgG at 1300, 625 and 312.5 pg. It is clear that the addition of 200 mM Boric acid slows down signal decay as compared to formulations containing 50 and 100 mM Boric acid.

The formulation containing sodium orthovanadate (Na₃VO₄) was prepared and tested on a Rabbit IgG dot blot. Two (2) separate solution A's were prepared, which comprised 10 mM Luminol, 0.8 mM BIPCA, 400 mM Tris, 200 mM Boric acid, and (i) 0 mM Na₃VO₄, or (ii) 1 mM Na₃VO₄, respectively.

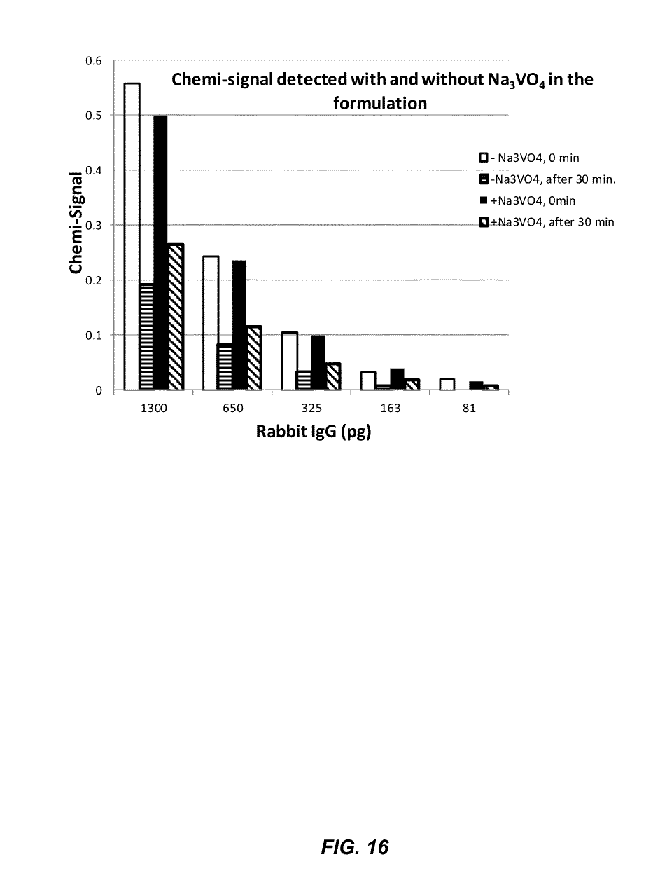

Solution B contains 8 mM NaBO₃ in 50 mM sodium acetate (pH 5.1). FIG. 16 displays the signal and its remaining after 30 min when blots were incubated with substrates with and without Na₃VO₄. Clearly, the Na₃VO₄ plays role in stabilizing the signal.

### Example 12

### A Pyridine Derivative Improves Chemiluminescence Substrates Performance

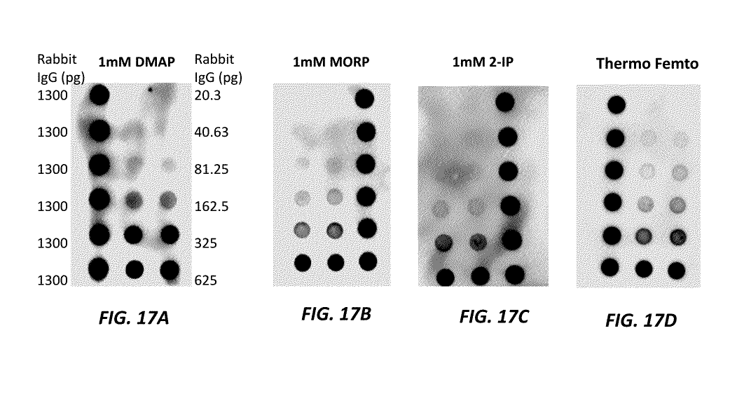

Visual detection limit (VDL) of Rabbit IgG on dot blot has been used to compare substrate performance. SuperSignal™ West Femto Maximum Sensitivity Substrate (Cat#34095) was used as a reference control (FIG. 17D). In this example, three formulations consisting of 4-(dimethylamino)pyridine (DMAP) (FIG. 17A), 4-morpholinopyridine (MORP) (FIG. 17B) and 2-(9-1H-imidiazole-2-yl)pyridines (2-IP) (FIG. 17C) were prepared. The components of three (3) solution A's are 15 mM Luminol, 0.8 mM BIPCA, 400 mM Tris, 200 mM Boric acid, 2% EG, 0.05% Triton X-100, 1 mM Na₃VO₄, with (i) 1 mM DMAP, or (ii) 1 mM MORP or (iii) 1 mM 2-IP (pH 10.8), respectively. Solution B contained 8 mM NaBO₃ in 50 mM sodium acetate (pH 5.1). FIG. 17A-C show the images of dot blots treated with four substrates. The pyridine derivatives containing substrates reached the comparable Visual detection limit (VDL) of SuperSignal™ West Femto Maximum Sensitivity Substrate.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A formulation for chemiluminescence, the formulation comprising a) a first part and b) a second part:
a) the first part comprising:
i) a diacylhydrazide selected from the group consisting of luminol, isoluminol or a luminol derivative;
ii) a phenol enhancer;
iii) optionally one or more co-enhancers;
iv) one or more stabilizers; and
b) the second part comprising:
v) an oxidant.

2. (canceled)

3. The formulation of claim 1, wherein i) the diacylhydrazide is luminol.

4. The formulation of claim 1, wherein ii) the phenol enhancer is a member selected from the group consisting of 4-indophenol, 4-iodophenol, 4-(3-thienyl)phenol, 4-(1-pyrrolyl)phenol, 4-(4-tolyl)phenol, 4-carboxy-4-hydroxybiphenol or 4′-hydroxy-[1,1′-biphenyl]-4-carboxylic acid (BIPCA), 4-bromo-4-hydroxyphenol, (E)-3-(4-hydroxyphenyl)acrylic acid, 4-(1H-imidazol-1-yl)phenol, 2-(4′-hydroxy-[1,1′-biphenyl]-4-ylcarboxamido)ethanesulfonic acid, 3-(4′-hydroxy-[1,1′-biphenyl]-4-ylcarboxamido)propane-1-sulfonic acid, 4′-hydroxy-[1,1′-biphenyl]-4-sulfonic acid, 5-hydroxy-1-naphthoic acid, 2-hydroxybenzoic acid, benzo[d]oxazol-6-ol, benzo[d]thiazol-6-ol, and (E)-4-hydroxy-3-(3-(4-hydroxyphenyl)acryloyl)-2H-chromen-2-one, 4-(1,2,4-triazol-1-yl) phenol, 4-phenylphenol, 4-(4,5-diphenyl-1H-imiazole)phenol and 4-(2-methyl-4-thiazolyl)phenol.

5. The formulation of claim 4, wherein ii) the phenol enhancer is 4-carboxy-4-hydroxybiphenol or 4′-hydroxy-[1,1′-biphenyl]-4-carboxylic acid (BIPCA).

6. The formulation of claim 1, wherein v) the oxidant is a member selected from the group consisting of a perborate and a peroxide.

7-12. (canceled)

13. The formulation of claim 1, wherein the one or more stabilizers is a member selected from the group consisting of a Lewis acid, a tertiary amine, a urea, ascorbic acid, a thiol and a pyridine derivative.

14. The formulation of claim 13, wherein the one or more stabilizers is a Lewis acid.

15. The formulation of claim 14, wherein the Lewis acid is a member selected from the group consisting of zinc acetate, zinc bromide, scandium triflate, sodium orthovanadate and boric acid.

16. The formulation of claim 14, wherein the Lewis acid further comprises a tertiary amine.

17. The formulation of claim 1, wherein the one or more stabilizers is a urea.

18. The formulation of claim 17, wherein the urea is dibenzylurea.

19. The formulation of claim 17, wherein the urea further comprises a tertiary amine or thiol.

20-21. (canceled)

22. The formulation of claim 1, wherein the one or more stabilizers is ascorbic acid.

23. The formulation of claim 17, wherein the urea formulation further comprises ascorbic acid.

24. The formulation of claim 17, wherein the urea formulation further comprises a co-enhancer of a transition metal salt.

25. The formulation of claim 24, wherein the transition metal salt is a member selected from the group consisting of Pd(0), Pd(II), Rh(III), Re(III), Ru(III), Ni(II), Mn(II), Mn(III) and combination thereof.

26. The formulation of claim 13, wherein the one or more stabilizers is a pyridine derivative.

27. The formulation of claim 26, wherein the pyridine derivative is a member selected from the group consisting of 4-(dimethylamino)pyridine (DMAP), 4-morpholinopyridine (MORP) and 2-(9-1H-imidiazole-2-yl)pyridines (2-IP).

28. A method for producing a chemiluminescence reaction, said method comprising:
admixing a first part of a formulation comprising:
i) a diacylhydrazide selected from the group consisting of luminol, isoluminol or a luminol derivative;
ii) a phenol enhancer and optionally a co-enhancer,
iii) one or more stabilizers, together with a second part comprising;
iv) an oxidant together with a catalyst to produce a chemiluminescence reaction and emit light.

29-55. (canceled)

56. A kit for chemiluminescence, the kit comprising a) a first part and b) a second part:
a) the first part comprising:
i) a diacylhydrazide selected from the group consisting of luminol, isoluminol or a luminol derivative;
ii) a phenol enhancer;
iii) optionally one or more co-enhancers;
iv) a stabilizer; and
b) the second part comprising:
v) an oxidant and instructions for use.

57-83. (canceled)
